(19) United States
(12) Patent Application Publication
(10) Pub. No.: US 2012/0053657 A1
(43) Pub. Date: Mar. 1, 2012

(54) IMPLANT RECHARGING

(76) Inventors: John Parker, Roseville (AU); Peter Single, Lane Cove (AU); Peter Ayre, North Willoughby (AU)

(21) Appl. No.: 13/222,833

(22) Filed: Aug. 31, 2011

(30) Foreign Application Priority Data
Aug. 31, 2010 (AU) 2010903899

(51) Int. Cl. A61N 1/378

(52) U.S. Cl. 607/61

(57) ABSTRACT

Charging a battery of an implanted device involves positioning an external charging coil proximal to a charging locale. An implant recipient and the implanted device are expected to occupy the charging locale from time to time. The charging coil has a coil area which is significantly larger than a coil area of an implanted coil, and is configured to produce an electromagnetic field throughout the charging locale. When an implant recipient is within the charging locale, the external charging coil is driven with a signal which transmits electromagnetic power from the charging coil to the implanted coil.

### RELATED APPLICATIONS

This application claims the benefit of Australian Provisional Patent Application No. 2010903899, filed Aug. 31, 2010, and incorporates by reference the entire disclosure thereof.

### FIELD OF THE INVENTION

The present invention relates to active implantable devices such as neuro-stimulating devices, and in particular the present invention provides components and a system for recharging such devices.

### BACKGROUND OF THE INVENTION

Active implantable medical devices usually consist of an electronics module and an interface mechanism to tissue. Current implantable neuro-stimulators consist of a hermetically sealed electronics module which may contain one or more batteries, and which is interfaced to an electrode system.

A schematic of a typical spinal cord stimulation (SCS) system is shown in FIG. 1. The SCS system consists of an IPG (Implantable pulse generator) 3 and electrode(s) 8 coupled to the IPG and designed to be inserted into the epidural space of the spinal cord. SCS systems employ a fixed number of electrodes, typically numbering in the range of 2 to 20 electrodes. Each electrode is contacted by a wire and terminated at corresponding feed-through connection 4 on an implant.

The power source 24 can be a rechargeable battery, and so a means must be developed to charge the battery. This is generally accomplished by coupling an external transmitting coil with a tuned receiving coil. The coupled coils provide a means to transmit power across the skin. The SCS system of FIG. 1 thus includes an inductive transmitter 21 designed to provide power to an inductive receiver in order to charge an implanted rechargeable power source 24. A second RF link formed by communication between an external transceiver 11 and internal transceiver 13 is used to send data back and forth from the external control unit to the implant. The data is used to set parameters within the device and receive data from the device (for instance impedance telemetry).

Similar system architectures are often used not only for SCS systems as shown in FIG. 1 but also for cochlear implants, deep brain stimulators, and the like.

Currently available active implant devices such cochlear implants, deep brain stimulators, and spinal column stimulators, for space reasons usually must have the implanted IPG somewhat distal from the electrode stimulus sites and this has meant that the IPG location is chosen partly to optimize transcutaneous power and/or data transfer over links 12 and 22. In most devices the transmitting coil is approximately the same size as the receiving coil and is configured to be pressed against the user's skin so that the coil separation is about the same as the skin thickness and significantly less than the diameter of either the implanted or external coil. Similarly sized and closely positioned coils are desirable in order to effect magnetic coupling with a coupling coefficient as close to 1 as possible.

However, to effect magnetic coupling with a high coefficient of coupling requires accurate positioning and alignment of the external coil relative to the internal coil. Some devices such as cochlear implants utilize a magnet to align the external coil relative to the implanted coil.

U.S. Pat. No. 6,047,214 discloses a system in which the implanted coil is not immediately beneath the skin, and teaches that power transfer can be effected by using multiple external solenoids and coils to steer the net magnetic vector towards the implanted coil in order to improve the coefficient of coupling. This requires knowledge of the location of the implanted coil relative to the external charging coils.

U.S. Pat. No. 7,231,254 teaches the use of 2 or 3 external charging coils to recharge an implant when in close proximity to the user's head, the coils being orthogonally arranged in a head rest so that at least one of the coils will enjoy a high coefficient of coupling.

US Patent Application Publication No. 2007/0129767 provides implant recharging by use of a plurality of external coils, with charging being optimized by a coil selection circuit which selects which coil to use based on which coil is experiencing the highest coefficient of coupling for effective power transfer.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY OF THE INVENTION

According to a first aspect the present invention provides a method for charging a battery of an implanted device, the method comprising:
positioning an external charging coil proximal to a charging locale which an implant recipient and the implanted device is expected to enter from time to time, the charging coil having a coil area which is significantly larger than a coil area of an implanted coil, and the charging coil being configured to produce an electromagnetic field throughout the charging locale; and
at least at a time when an implant recipient is within the charging locale, driving the external charging coil with a signal which transmits electromagnetic power from the charging coil to the implanted coil.

According to a second aspect the present invention provides a charging device for charging a battery of an implanted device, the charging device comprising:
an external charging coil for positioning proximal to a charging locale which an implant recipient and the implanted device is expected to enter from time to time, the charging coil having a coil area which is significantly larger than a coil area of an implanted coil, and the charging coil being configured to produce an electromagnetic field throughout the charging locale
a signal generator for generating a charging signal to drive the external charging coil in order to deliver power electromagnetically from the external charging coil to the implanted device at least at a time when an implant recipient is within the charging locale.

The charging coil may have one turn or multiple turns. The coil area of the charging coil is preferably at least five times the coil area of the implanted coil, more preferably at least ten times the coil area of the implanted coil, and more preferably at least fifty times the coil area of the implanted coil. For example, the external coil may have a diameter of substantially 20 centimetres or greater, while the implanted coil will typically have a diameter of about three centimetres or less. In preferred embodiments the coil area of the charging coil is sufficiently large to permit adequate energy density to be produced within the charging locale to permit sufficient power transfer to effect battery charging, while maintaining peak power intensities below levels prescribed for consumer exposure.

The external charging coil may be energised only at times when it is determined that the implanted device is present within the charging locale, for example the charging coil may be activated by a pressure sensor, metal detector, motion detector or other sensor configured to sense the presence of the implant recipient and implanted device at the charging locale. Alternatively, the external charging coil may illuminate the charging locale with electromagnetic power irrespective of the presence of the implanted device.

By illuminating the entire charging locale with electromagnetic energy from the charging coil, the present invention eliminates the need to precisely align internal and external coils, or the need to provide a plurality of external coils, in order to effect power transfer. Effectively, the present invention provides for a system in which the coefficient of coupling between the charging coil and the implant coil is deliberately reduced, in order to relax coil alignment constraints.

The present invention recognizes that with improved implant component design and miniaturization, the implanted controller and its coils/transceivers will increasingly be positioned at sites to maximize therapeutic benefits, to the detriment of wireless transcutaneous link performance. As such solutions develop, tightly coupled coils having a known transfer function will become less feasible as a means for wireless data and power transfer.

The charging process may be passively initiated, for example upon the external device sensing proximity of the implanted device, or sensing the presence of a human. For example a mattress or seat-mounted charging device may have a pressure sensor which initiates charging. The sensor may be a temperature sensor, metal detector, or an electromagnetic coil communicating with an implant.

The external device may be mounted in an everyday article regularly used by the implant recipient, for example a chair, a bed, a mattress, a pillow, furniture, clothing, or a car seat. The external device may be mounted in a fixed structure in relation to which the implant recipient is often in proximity, such as a wall, floor or ceiling of the implant recipient's home or workplace.

The charging locale is preferably an everyday location in which the implant recipient and the implanted device can be expected to reside for sufficient time each day to effect sufficient battery charging. For example, the charging locale may be the space occupied by the implant recipient when sleeping, eating, working, or driving a vehicle.

The external coil may be positioned to illuminate the charging locale by being mounted within or upon a wall, ceiling or floor of a room within which the charging locale resides. Alternatively, the external coil may be positioned to illuminate the charging locale by being mounted within or upon furniture occupied by the implant recipient when present in the charging locale, such as an office chair, a dining table chair, a lounge chair, a mattress, a bed, or a seat of a car.

By selectively implementing the relative positions of the coils in a manner which effects a relatively low coefficient of coupling, embodiments of the present invention may thus provide for a larger coil spacing, and thus a longer charge range, than charging devices relying on maximising a coefficient of coupling. An increase charge range can be advantageous in permitting the external charging device to be powered by mains power, rather than battery power as is generally required for ambulatory or body-worn devices and the like brought close to the implant.

In some embodiments the implanted device may assess a charge state of the battery and communicate said charge state to an external device, with the external device determining from the charge state of the battery a suitable electromagnetic charging signal to be delivered by the external coil. The generated signal delivered by the or each external coil may be adapted in response to the assessed state of the implant battery in a number of ways. For example, the power of the delivered signal may be varied. Alternatively a frequency of the delivered signal may be varied. Variation of the delivered signal may be controlled in a manner to seek a desired rate of charge of the implant battery. In some embodiments the implanted device repeatedly assesses a charge state of the battery throughout recharging, and repeatedly communicates the charge state to the external device in order to repeatedly refine the recharging process.

The external charging coil may effect both delivery of power and may also receive the communication of the charge state of the battery from the implanted device. Alternatively, a separate external communications coil for communicating with the implant may be provided in addition to the charging coil.

The implanted device may be provided with a single coil operable to both receive power from the external charging coil and to wirelessly communicate with an external device. Alternatively, the implanted device may have a first coil for communicating with an external device and a second coil for receiving electromagnetic power from the external charging coil.

### BRIEF DESCRIPTION OF THE DRAWINGS

An example of the invention will now be described with reference to the accompanying drawings, in which:

FIG. 1 is a schematic of a typical spinal cord stimulation (SCS) system;

FIG. 2 illustrates a charging device and use thereof in accordance with a first embodiment of the invention;

FIG. 3 illustrates a charging device and use thereof in accordance with a second embodiment of the invention;

FIG. 4 illustrates a charging device and use thereof in accordance with a third embodiment of the invention;

FIG. 5 illustrates a charging device and use thereof in accordance with a fourth embodiment of the invention;

FIG. 6 illustrates a charging device and use thereof in accordance with a fifth embodiment of the invention; and

FIG. 7 is a functional block diagram of a recharging system in accordance with one embodiment of the present invention.

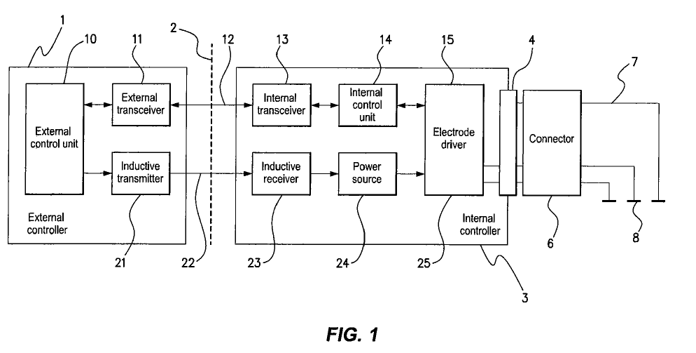

Corresponding reference characters indicate corresponding components throughout the drawings.

### DETAILED DESCRIPTION OF PREFERRED EMBODIMENTS

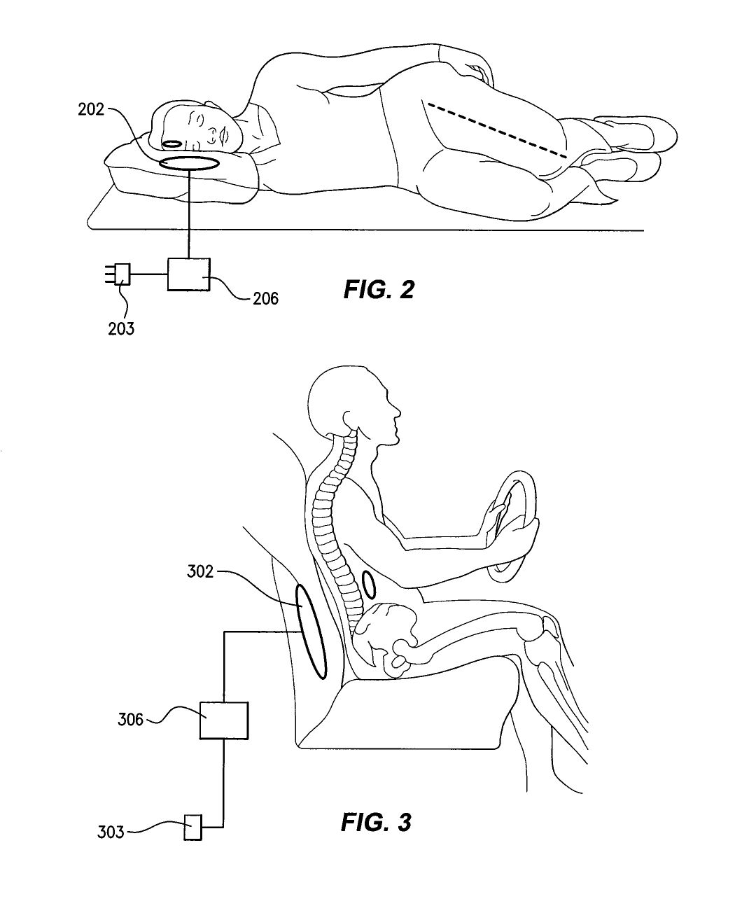

FIG. 2 illustrates a charging device and use thereof in accordance with a first embodiment of the invention. An external charging coil 202 is embedded within a pillow so as to charge the cranial implant when the implant recipient is lying down or sleeping. Notably, the external coil 202 is significantly larger in diameter than the implant coil which, while reducing the coefficient of coupling, increases the size of the locale illuminated by the charging coil thus easing requirements for accurate alignment and close coil spacing and permitting such an implementation. A charging controller 206 generates a suitable signal to drive the coil 202 and uses mains power 203.

FIG. 3 illustrates a charging device and use thereof in accordance with a second embodiment of the invention. An external charging coil 302 is embedded within a back rest of a car seat so as to charge an abdominal or spinal implant when the implant recipient is driving. Notably, the external coil 302 is significantly larger in diameter than the implant coil, again easing requirements for accurate alignment and close coil spacing and permitting such an implementation. A charging controller 306 generates a suitable signal to drive the coil 302 and uses power derived from the car's battery 303.

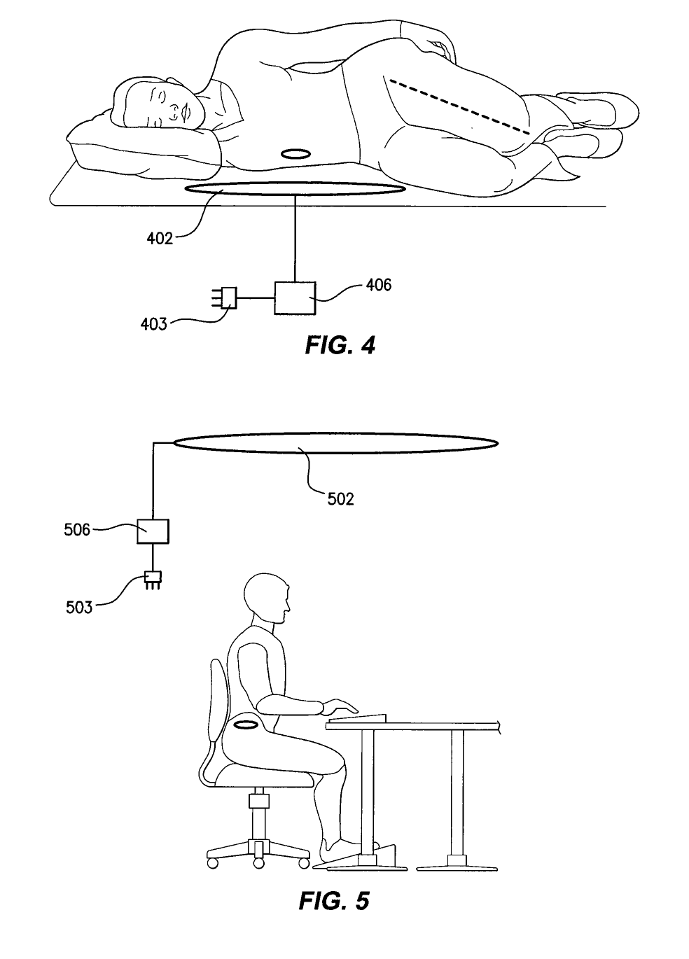

FIG. 4 illustrates a charging device and use thereof in accordance with a third embodiment of the invention. An external charging coil 402 is embedded within a mattress so as to charge an abdominal or spinal implant when the implant recipient is lying down on the mattress. Notably, the external coil 402 is significantly larger in diameter than the implant coil, easing requirements for accurate alignment and close coil spacing and permitting such an implementation. A charging controller 406 generates a suitable signal to drive the coil 402 and uses mains power 403.

FIG. 5 illustrates a charging device and use thereof in accordance with a fourth embodiment of the invention. An external charging coil 502 is embedded within a ceiling above a work station so as to charge an abdominal or spinal implant when the implant recipient is occupying the work station. Notably, the external coil 502 is significantly larger in diameter than the implant coil, easing requirements for accurate alignment and close coil spacing and permitting such an implementation even though the coil 502 may be two metres or more above the implant. A charging controller 506 generates a suitable signal to drive the coil 502 and uses mains power 503.

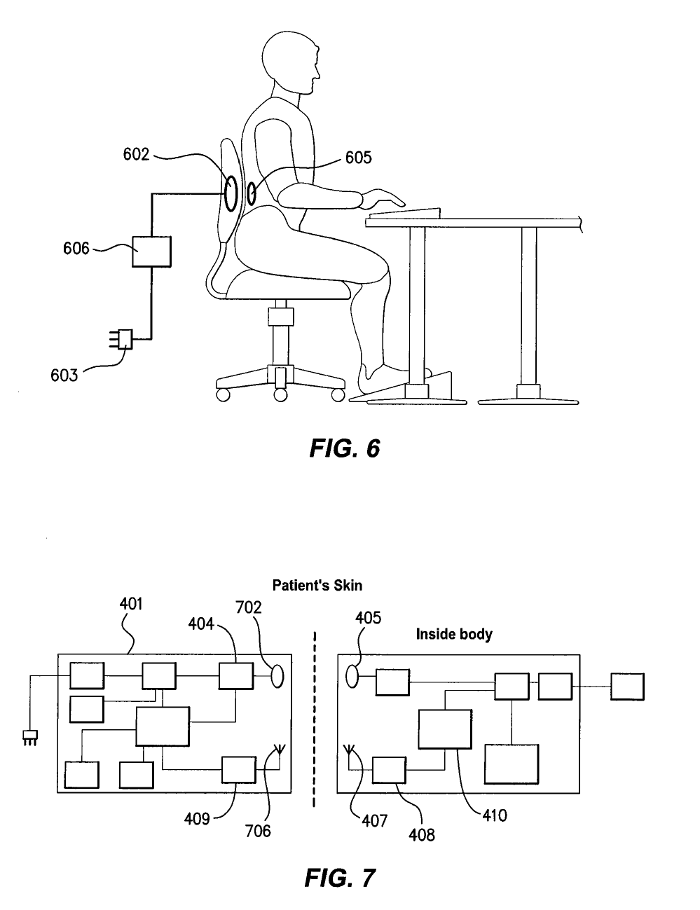

FIG. 6 illustrates a charging device and use thereof in accordance with a fifth embodiment of the invention. An external charging coil 602 is embedded within a back rest of an office chair at a work station so as to charge an abdominal or spinal implant when the implant recipient is occupying the office chair. Notably, the external coil 602 is significantly larger in diameter than the implant coil 605, easing requirements for accurate alignment. A charging controller 606 generates a suitable signal to drive the coil 602 and uses mains power 603.

In these embodiments, the tuned transmitting coil has a large surface area which ensures the coil illuminates a large charging locale, meaning that the recharging process will not be significantly compromised should transverse, coronal or sagittal planar movement of the body occur relative to the transmitting coil, provided the implant coil remains generally within the charging locale.

A functional block diagram of a system in accordance with one embodiment of the invention is shown in FIG. 7. The power amplifier module 401 consists of a tuned coil 702 which is driven by a driver 404 at a frequency that is resonant to both the transmitting coil 702 and the receiving tuned coils 405 of the implant. At the beginning of the charge cycle of the implant, the implant transmits a signal via a separate RF link using separate link antennae 706, 407 and corresponding transceivers 408, 409. The implant and external controller synchronise information as necessary to understand the state of charge of the implanted battery. During this synchronization where data is transferred from implant to external charging unit, information other than the status of the battery such as information which relates to the use of the device or settings may be transferred. The recharging module 401 may be equipped with a user interface which alerts the recipient of the status of the charging system or implant. Such user interface may consist of any or all of audible or visual means.

A processor within the implant 410 manages the state of charge of the battery and communicates via the RF link with the recharging unit. The parameters in the system can be set to control the behaviour of the system with respect to feedback to the user. The system can be set to indicate when charging is complete via audible or visual means. The therapy can be maintained during the charging cycle.

These embodiments of the invention thus relate to a charging device which uses a large external induction charging loop for charging the implanted device. The configurations of the described embodiments provide mismatched size of the charging and implant coils, and also provide for a relatively large coil separation of tens or hundreds of centimeters, thus effecting a relatively low coefficient of coupling between the charging coil and the implant coil. Nevertheless, as the present invention permits for the external charging coil to be located at a distance from the implant, the external device would typically have a sufficient power source, such as mains power or a car battery, with which to generate the desired field strength without being constrained to small battery power as is the case for most body-worn chargers. Accordingly, the present invention recognises that reducing the coefficient of coupling can be accommodated by increasing the overall field strength to ensure that sufficient field couples with the implanted device to enable recharging, even if a large portion of the field energy is not harnessed by the implanted device.

Some embodiments of the present invention recognise that it is not always simple or easy to locate the implant site to ensure that a small tightly coupled coil is placed directly over the implant site. This difficulty only increases with reducing size of implanted devices as is occurring for example to facilitate implant positioning very close to the site of stimulation. Additionally, for the specific case of a spinal cord stimulator located on the spine an individual may not have the dexterity or flexibility to reach around their back to accurately place a charging coil.

Another alternative embodiment is a general purpose recharging fabric, containing a suitable charging coil, which can be laid over the patient such as when in a chair or bed. Similarly, the external charging coil may be integrated into garments.

In embodiments where there is a remote control that is used to control the implanted device, the charging system may be switched on and off by use of the implant device remote control. For example, in such embodiments the external charging device may detect the proximity of the implanted device by any suitable method, such as sensing pressure, temperature, RF etc. The external charging device then wirelessly interrogates the remote control and, if the remote control is close enough to communicate and it is set to manual charge mode, then the remote control indicates to the user that it is now possible to charge. The user may then use the remote control to initiate the charging cycle.

In other embodiments, the external recharging device may have the capacity to turn on and off automatically whenever it detects the proximity of both the implanted device and the remote control. For example the charger may detect the proximity of the implanted device by any suitable method, then upon detection may wirelessly interrogate the remote control. If the wireless interrogation establishes that the remote control is close enough to communicate, and the remote control is set to an auto-charge mode, then the charging cycle begins.

Embodiments of the invention may be applied to recharge implant devices used for deep brain stimulation (DBS) or early chronic cerebellar stimulation (CCS) for the treatment of pain and movement disorders. For example, some embodiments of the invention may be employed to effect one or more of: DBS for Parkinson's treatment; DBS of the internal pallidum or subthalamic nucleus to treat upper limb akinesia in Parkinson's disease; DBS for treatment of medication-refractory idiopathic generalized dystonia, DBS in treatment of Spasticity and Seizures; bilateral DBS of the internal pallidum and the subthalamic nucleus to improve motor function, movement time, and force production; DBS for the treatment of pain such as failed back syndrome, peripheral neuropathy, radiculopathy, thalamic pain, trigeminal neuropathy, traumatic spinal cord lesions, causalgic pain, phantom limb pain, and carcinoma pain; and DBS for treatment of essential tremor, for example.

Thus, while the benefits and applications of these embodiments are described for devices for spinal cord stimulation, deep brain stimulation and cochlear implants, the present invention is not limited to such applications.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method for charging a battery of an implanted device, the method comprising:
positioning an external charging coil proximal to a charging locale which an implant recipient and the implanted device is expected to enter from time to time, the charging coil having a coil area which is significantly larger than a coil area of an implanted coil, and the charging coil being configured to produce an electromagnetic field throughout the charging locale; and
at least at a time when an implant recipient is within the charging locale, driving the external charging coil with a signal which transmits electromagnetic power from the charging coil to the implanted coil.

2. The method of claim 1 wherein the external charging coil is energised only at times when it is determined that the implanted device is present within the charging locale.

3. The method of claim 2 wherein the charging coil is activated in response to a sensor configured to sense the presence of the implant recipient and implanted device at the charging locale.

4. The method of claim 2 wherein the charging coil is activated in response to a user input.

5. The method of claim 1 wherein the external charging coil illuminates the charging locale with electromagnetic power irrespective of the presence of the implanted device.

6. The method of claim 1 wherein the charging coil is positioned at least about two wavelengths from the charging locale.

7. The method of claim 1 further comprising the implanted device assessing a charge state of the battery and communicating said charge state to an external device, with the external device determining from the charge state of the battery a suitable electromagnetic charging signal to be delivered by the external coil.

8. A charging device for charging a battery of an implanted device, the charging device comprising:
an external charging coil for positioning proximal to a charging locale which an implant recipient and the implanted device is expected to enter from time to time, the charging coil having a coil area which is significantly larger than a coil area of an implanted coil, and the charging coil being configured to produce an electromagnetic field throughout the charging locale
a signal generator for generating a charging signal to drive the external charging coil in order to deliver power electromagnetically from the external charging coil to the implanted device at least at a time when an implant recipient is within the charging locale.

9. The device of claim 8 wherein the coil area of the charging coil is at least five times the coil area of the implanted coil.

10. The device of claim 9, wherein the coil area of the charging coil is at least ten times the coil area of the implanted coil.

11. The device of claim 10, wherein the coil area of the charging coil is at least fifty times the coil area of the implanted coil
